(19) United States
(12) Patent Application Publication
(10) Pub. No.: US 2008/0214844 A1
(43) Pub. Date: Sep. 4, 2008

(54) Icaritin and desmethylicaritin as anti-cancer agents

(76) Inventors: Nan Zhang, The Woodlands, TX (US); Stuart K. Calderwood, Brookline, MA (US)

Correspondence Address: LAW OFFICES OF J.D. GERAIGERY, P.C., 56 CREIGHTON STREET, CAMBRIDGE, MA, 02140, US

(21) Appl. No.: 11/657,214

(22) Filed: Jan. 23, 2007

(51) Int. Cl. C07D 311/30

(52) U.S. Cl. 549/400

(57) ABSTRACT

The antiproliferative effects of Icaritin or Desmethylicaritin on cancer cell lines, both in vitro, and in vivo, are disclosed. Experimental data show that Icaritin and Desmethylicaritin dramatically inhibit the growth of most malignant cells. In addition, both Icaritin and Desmethylicaritin have significant Anti-agiogenesis properties, inhibiting or eliminating entirely the development of new malignant cells. Further, no obvious side effects including nausea, hair loss or body weight loss were found in the animals treated with Icaritin or Desmethylicaritin, making both highly effective anti cancer drugs.

### BACKGROUND OF THE INVENTION

Epimedium is a pungent ornamental herb found in Asia and the Mediterranean. The Chinese call Epimedium Yin Yang Huo, which loosely means “Licentious Goat Plant.” The herb was named Epimedium because it is similar to a plant found in the ancient Asian kingdom of Media, now a part of Iran. Epimedium is a genus of many related plant species and some are used for medicinal purposes, including Epimedium sagittatum, Epimedium brevicornum, epimedium grandiflorum, and Epimedium koreanum. Epimedium sagittatum is an important traditional Chinese herbal medicine wildly used as a tonic, and antiheumatic in China, Japan and Korea, and has been proved effective against osteoporosis, cardiovascular diseases and cancer diseases.

Icariin (ICA)(molecule weight=676), a flavonol glycoside, is a major compound of Epimedium sagittatum. Icariin is a kind of new biological response modifier (BRM) and differentiational agent which has been found effective in treating a broad range of malignant growths. In order to further elucidate the reversion of malignant phenotypes of tumor cells and the mechanism of its action, highly metastatic human lung cancer cells (PG) were treated with ICA in vitro. The results showed that ICA could influence the distribution of PG cells cycle and reduce S phase. Moreover, ICA increased the level of cAMP in PG cells, reduced the level of cGMP and increased the cAMP/cGMP ratio. On the other hand, ICA decreased PG cells adhesive ratio to laminin substrate and decreased the ability of invasion or migration. ICA was also found to enhance membrane fluidity of PG and increase the expression of membrane HLA-ABC antigen. These data demonstrate that ICA maybe a kind of effective anticancer drug. However, in the other studies, ICA showed no inhibition of either the hepatoma or leukemia cell lines.

ICA was extensively biotransformed and converted to at least three metabolites: Icariside II (molecule weight=514), Icaritin (molecule weight=368), and Desmethylicaritin (molecule weight=354). Icaritin and Desmethylicaritin were found to act as week phytoestrogens and their estrogenic effects were mediated by the estrogen receptor. The inventor has found that Icaritin and Desmethylicaritin broadly inhibit tumor cell growth of many types of human tumor cell lines and further induce the tumor cell apoptosis. In three human cancer xenograft model studies, Icaritin was demonstrated to be an effective anti cancer agent by strongly inhibiting human lung, colon and prostate cancer cell growth in vivo. Icaritin and Desmethylicaritin have huge potentials to be developed as novel and broad anti cancer drugs.

Epimedium sagittatum, originally used to isolate Icaritin and Desmethylicaritin, is an important traditional Chinese herbal medicine wildly used as a tonic, and antiheumatic in China, Japan and Korea, and has been proved effective against osteoporosis, cardiovascular diseases and cancer diseases.

### SUMMARY OF THE INVENTION

Icaritin and Desmethylicaritin broadly inhibit human tumor cell lines, but not normal cell lines. 26 cancer cell lines were grown in normal growth medium in the presence of different concentrations Icaritin or Desmethylicaritin. After culture for 3 days, the cell viability was then measured by an MTT assay. The results, summarized in Table 1, below, showed that most human tumor cells were sensitive to Icaritin and Desmethylicaritin. Some EC50s were lower than 10 uM for these two compounds. Furthermore, it was found that estrogen independent human breast cancer cells were more sensitive to Icaritin or Desmethylicaritin than estrogen dependent human breast cancer cells. Whereas, normal human mammary gland epithelial cells (MCF10A) were not sensitive to Icaritin and Desmethylicaritin. These cells grew well when treated with these two compounds at a concentration of 50 uM.

| | |
|---|---|
| Human tumor | IC50 (uM) |

| cell lines | Cell types | Icaritin | Desmethylicaritin |
|---|---|---|---|
| LNCAP | Prostate | 5.3 | 6.1 |
| D145 | Prostate | 9.3 | 9.8 |
| PC3 | Prostate | 9.4 | 10.6 |
| HCT-116 | Colon | 6.4 | 6.9 |
| Widr | Colon | 10.6 | 11.3 |
| HT29 | Colon | 15.3 | 16.8 |
| LoVo | Colon | 10.3 | 10.5 |
| CCL-225 | Colon | 8.8 | 8.9 |
| CCL-247 | Colon | 16.5 | 17.6 |
| NCI-H23 | Lung | 6 | 6.5 |
| A549 | Lung | 10.6 | 10.7 |
| MDA-MB-231 | Breast | 5.3 | 6.5 |
| MDA-MB-435 | Breast | 10.2 | 12.7 |
| AU-565 | Breast | 7.9 | 8.9 |
| BT-549 | Breast | 8 | 9.6 |
| MCF-7 | Breast | 18.2 | 24.8 |
| Caki-1 | Kidney | 7 | 7.5 |
| ACHN | Kidney | 9.5 | 9.7 |
| 786-O | Kidney | 10.5 | 10.9 |
| SN12C | Kidney | 15.8 | 15.9 |
| SKOV3 | Ovary | 9.8 | 10.4 |
| IGROV1 | Ovary | 10.9 | 11.2 |
| Mid PaCa-2 | Pancreas | 8.1 | 8.6 |
| U-251 | Glioblastoma | 7.1 | 7.3 |
| SK-MEL-5 | Skin | 9.9 | 10.8 |
| G-361 | Skin | 6.9 | 7.4 |
| Raji | Lymphoma | 5.4 | 5.2 |
| Jurkat T cell | Leukemia | 5.1 | 4.9 |
| MCF-10a | Normal breast | >50 | >50 |
| | epithelial cells | | |

To investigate the ability of Icaritin and Desmethylicaritin to inhibit lipogenesis, LNCaP prostate cancer cells were treated with different concentrations of Icaritin and Desmethylicaritin for 5 h, and incorporation of 2-¹⁴C-labeled acetate into cellular lipids was quantitated. Both of Icaritin and Desmethylicaritin inhibited lipogenesis by more than 50% at a concentration of 0.5 uM. Higher concentrations further reduced lipogenesis in a dose-dependent way. A further decline was observed after 24 h of exposure.

Icaritin and Desmethylicaritin decrease Lipogenesis in Prostate and Breast Cancer cells via inhibition of fatty acid synthase (FAS) Activity. Protein extracts from LNCaP cells, which had been ¹⁴C-labeled with malonyl-CoA, were pretreated with these two compounds. A concentration of 0.5 μM Icaritin or Desmethylicaritin reduced the in vitro enzymatic FAS activity to less than 50%. Western blot analysis for FAS revealed that Icaritin and Desmethylicaritin did not influence FAS protein levels in LNCaP cells, thereby confirming that inhibition of lipid synthesis was the direct result of inhibition of enzymatic FAS activity and was not caused by decreased FAS expression. Consistent with their FAS inhibitory effect, inhibition of lipogenesis by Icaritin and Desmethylicaritin affected the synthesis of phospholipids (including phosphatidylcholine, phosphatidylethanolamine, phosphatidylinositol, phosphatidylserine, and sphingomyelin) and of triglycerides in LNCaP cells and MDA-MB-231 cells. As previously observed for EGCG and cerulenin, Icaritin and Desmethylicaritin also decreased the synthesis of cholesterol in LNCaP and MDA-MB-231 cells. Cholesterol synthesis, however, accounts for less than 10% of the total lipogenic activity in LNCaP and MDA-MB-231 cancer cells, whereas ˜75 and 15% of the lipogenic activity in these cells represents phospholipid and triglyceride synthesis, respectively.

Icaritin is a potent inhibitor of angiogenesis. Inhibition of human micro vessel endothelial cell (HMVECs) proliferation by Icaritin was investigated. HMVEC proliferation in response to Icaritin was determined using a modified method from previously described procedures. Briefly, HMVECs fed 1 day before use (with a fresh media exchange to obtain optimal growth conditions) were trypsinized and resuspended at 5×10⁴ cells/ml in EBM (Clonetics)+2% FBS+gentamicin. An equivalent number of cells (2.5×10³) in 50 μl cell suspension was plated nonconfluently onto each well of a 96-well plate and allowed to adhere for 4 h at 37° C. Serial Icaritin dilutions were prepared in this media at 3× concentration, of which 50 μl was added to the 100 μl cell suspension already in the wells, resulting in 150 μl 1× dilutions. The plate was incubated for up to 72 h at 37° C. The Cell Titer 96 Aqueous nonradioactive cell proliferation assay kit (Promega, Madison, Wis.), which contains a tetrazolium compound and phenazine methosulfate solution, was diluted according to the recommended protocol and added to each well (20 μl/well). The plates were incubated at 37° C. for 2 h until color development occurred. Absorbance was read at 490 nm on an ELISA plate reader with Microplate Manager (Bio-Rad, Richmond, Calif.). Proliferation curves for HMVECs were determined, and measurements were obtained and compared in the log proliferation phase, which was 3 days (t=72 h) for the conditions described. Serial dilutions of Icaritin, ranging from 100 nM to 30 uM were assayed. The data showed that Icaritin resulted in significant levels of inhibition of proliferation and inducing cell apoptosis in HUVECs and HMVECs at a concentration of 10 uM. The EC50 for Icaritin was 8.7 uM.

Endothelial cell tube formation in Matrigel is one measure of angiogenesis in vitro. The role of Icaritin in angiogenesis was assessed by determining the inhibition of HMVEC tube formation on Matrigel matrix plated onto 96-well plates. The HMVEC tube formation assay was performed as described previously. Briefly, 96-well plates were coated with 90 μL Matrigel (10 mg/mL, Collaborative Research) and incubated at 37° C. for 30 minutes to promote gelling. HMVECs (10,000 cells/sample) were resuspended in reduced growth medium (serum concentration 1% and 5 units/mL heparin) and added to each well with the indicated reagents in a final volume of 100 μL with different concentration of Icaritin. After 18 hours, the plates were fixed with Diff-Quik and at least four randomly selected fields per experimental condition were digitally recorded under bright-field illumination using a 10× objective. The mean additive length of all of the cords present in each optical field was measured using IPLab software and compared with controls using the Student's unpaired t test. The results showed that Icaritin completely inhibited the HMVEC tube formation at 10 uM. The EC50 of Icaritin to inhibit HMVEC tube formation was 5.8 uM.

We can make a conclusion that Icaritin is a potent inhibitor of angiogenesis that inhibits endothelial cell migration and tube formation.

### DESCRIPTION OF THE PREFERRED EMBODIMENT

Icariin, the precursor of Icaritin and desmehylicaritin was produced through the following procedure: Epimedium sagittatum was collected from the southern mountain area in China and ground into powder. The powder was extracted using 95% ethanol then further extracted with first, chloroform, then ethyl acetate, and finally, n-butanol. The acetic acetate and n-butanol extracted parts were loaded into separate silica gel chromatographic columns and eluted with CHCl₃-MeOH—HCOOH (15:1:0.5), CHCl₃-MeOH (8:2˜7:2) respectively.

Icaritin was derived from Icariin by hydrolysis as follows: The precursor, dissolved with ultrasound in aqueous methanol, was slowly added with stirring, to a solution of cellulose dissolved in 0.1 molar acetic acid buffer (pH=5). The mixture was incubated in a shaking water bath at 37° C. overnight to cleave the glucose and rhamnose conjugated on the precursor. Icaritin was then extracted using three equal volumes of ethyl acetate, which was evaporated to dryness in vacuo on a rotavapor.

Desmethylicaritin was derived from Icaritin by demethylation described as follows. In a flask with a drying tube filled with calcium chloride, a solution of Icaritin in freshly distilled dichloromethane was added to a solution of boron tribromide in dichloromethane and reacted for one hour at −80° C. The mixture was kept overnight, where it warmed to room temperature, then was shaken with water to hydrolyze excess boron tribromide and boron complexes. Desmethylicaritin was extracted using three equal volumes of ethyl acetate, which was then evaporated to dryness in vacuo on a rotavapor.

### DETAILED DESCRIPTION OF THE DRAWINGS

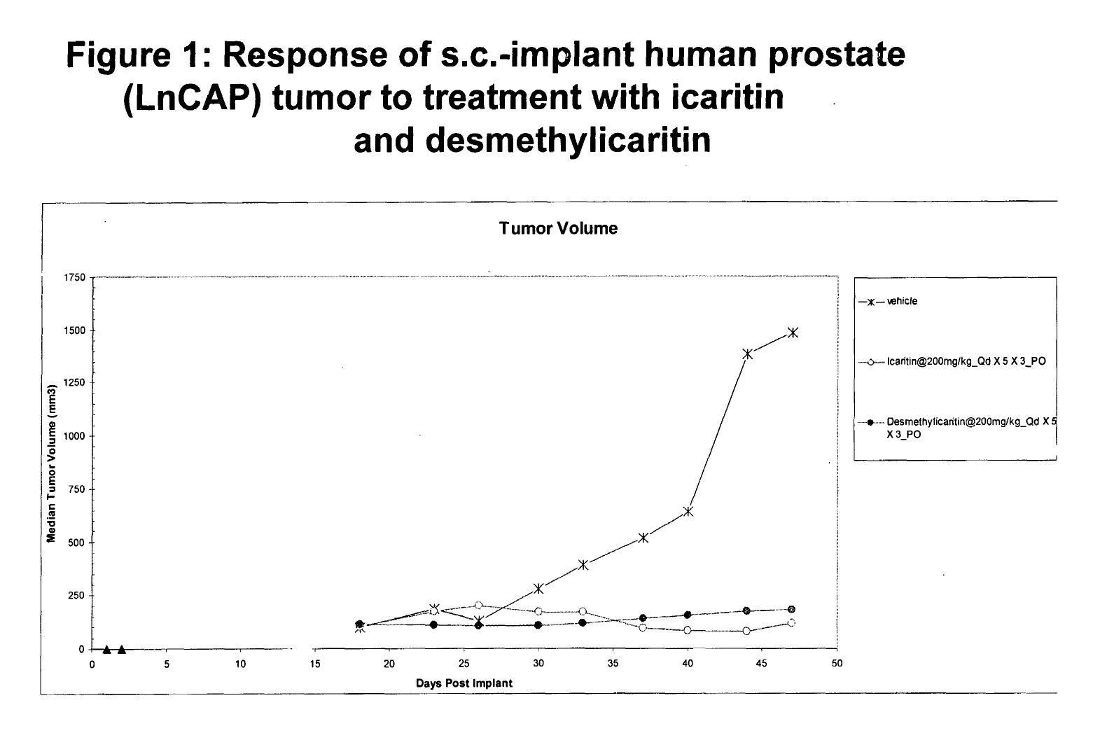

FIG. 1. Response of Human Prostate tumor to treatment with Icaritin and Desmethyliciarityn. The line connecting the X's shows the growth rate of tumors in the control case, the lines connecting the open circles show the growth of the tumor when treated with Icaritin and the lines connecting the solid circles show the growth of the tumor when treated with Desmethylicaritin

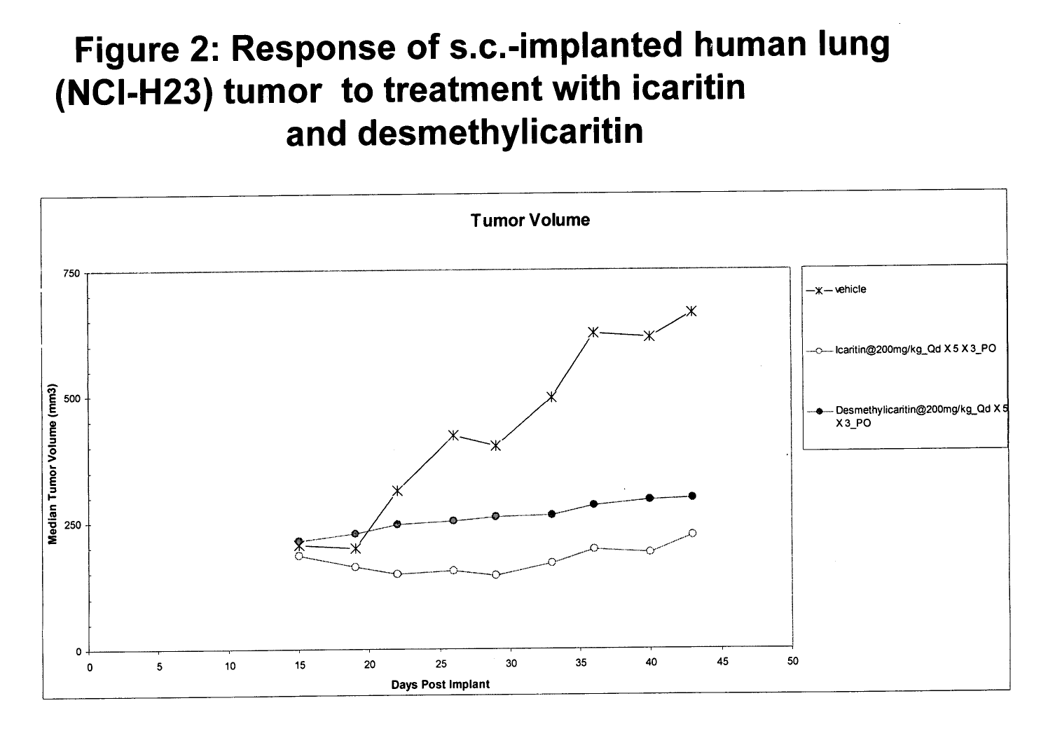

FIG. 2. Response of Human Lung tumor to treatment with Icaritin and Desmethyliciarityn. The line connecting the X's shows the growth rate of tumors in the control case, the lines connecting the open circles show the growth of the tumor when treated with Icaritin and the lines connecting the solid circles show the growth of the tumor when treated with Desmethylicaritin

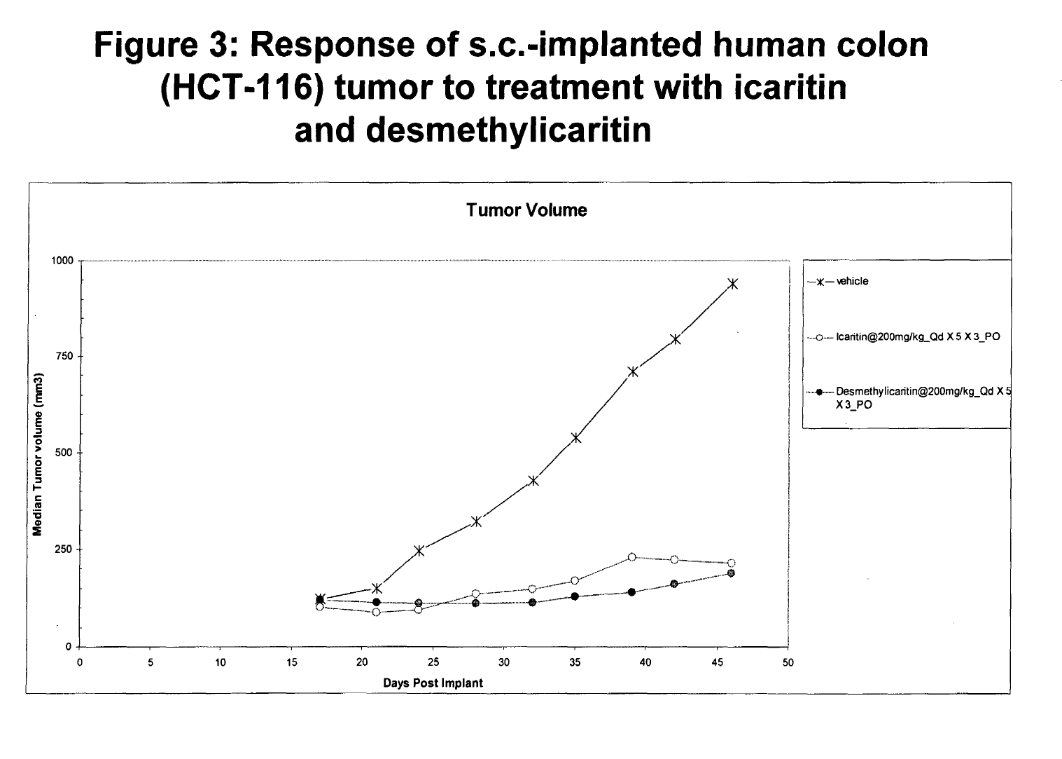

FIG. 3. Response of Human Colon tumor to treatment with Icaritin and Desmethyliciarityn. The line connecting the X's shows the growth rate of tumors in the control case, the lines connecting the open circles show the growth of the tumor when treated with Icaritin and the lines connecting the solid circles show the growth of the tumor when treated with Desmethylicaritin

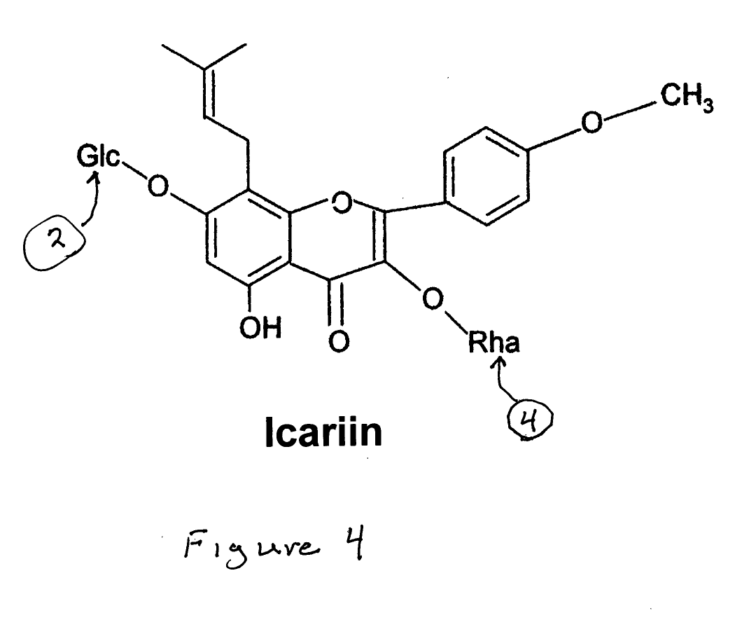

FIG. 4. A graphical representation of the Organic chemical known as Icariin (Molecular weight 676), where the symbols used have the usual interpretation. The moiety 2, shown as Glc, is Glucose and the moiety 4, shown as Rha is Rhamnose.

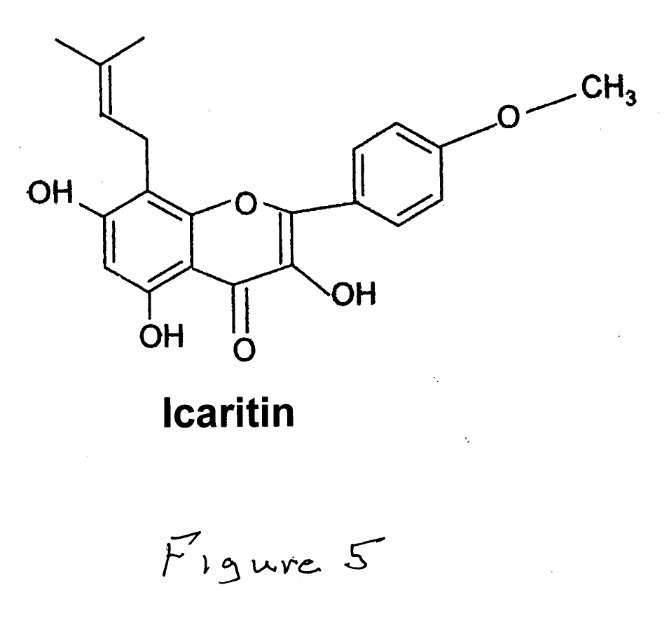

FIG. 5. A graphical representation of the Organic chemical known as Icaritin (Molecular weight 368), where the symbols used have the usual interpretation.

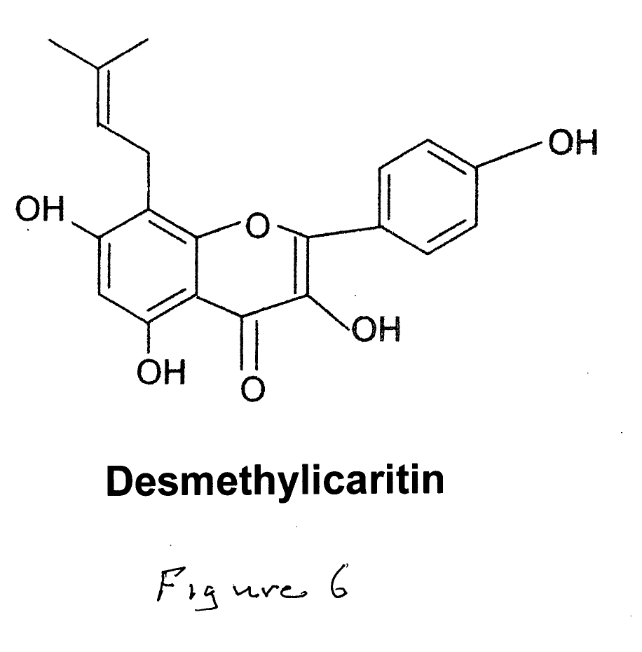

FIG. 6. A graphical representation of the Organic chemical known as Desmethylicaritin (Molecular weight 354), where the symbols used have the usual interpretation.

We claim:

1. A mammalian anti-cancer treatment for malignant cells consisting of the chemical known as Icaritin.

2. The anti-cancer treatment of claim 1 where the said malignant cells treated comprise prostate, colon, lung, breast, kidney, ovary, pancreas, glioblastoma, skin, lymphoma and leukemia.

3. The anti-cancer treatment of claim 2 wherein the mechanism of action is anti-angiogenesis.

4. A mammalian anti-cancer treatment for malignant cells consisting of the chemical known as Desmetylicaritin.

5. The anti-cancer treatment of claim 4 where the said malignant cells treated comprise prostate, colon, lung, breast, kidney, ovary, pancreas, glioblastoma, skin, lymphoma and leukemia.

6. The anti-cancer treatment of claim 5 wherein the mechanism of action is anti-angiogenesis.
